(19) United States
(12) Patent Application Publication
(10) Pub. No.: US 2025/0049087 A1
(43) Pub. Date: Feb. 13, 2025

(54) FOOD AND BEVERAGE PRODUCTS COMPRISING ASCOMYCETES

(71) Applicant: MYCOTECHNOLOGY, INC, Aurora, CO (US)

(72) Inventors: Anthony J. CLARK, Aurora, CO (US); Alan D. HAHN, Aurora, CO (US); Zheyuan GUO, Aurora, CO (US); Marina NADAL, Aurora, CO (US); Anthony WESTGATE, Aurora, CO (US); James Patrick LANGAN, Aurora, CO (US); Brooks John KELLY, Aurora, CO (US)

(73) Assignee: MYCOTECHNOLOGY, INC., Aurora, CO (US)

(21) Appl. No.: 18/930,855

(22) Filed: Oct. 29, 2024

Related U.S. Application Data
(63) Continuation of application No. 17/421,612, filed on Jul. 8, 2021, now abandoned.
(60) Provisional application No. 62/790,365, filed on Jan. 9, 2019.

(51) Int. Cl. A23L 27/30; A23L 2/60; A61K 9/00; A61K 36/062

(52) U.S. Cl. CPC A23L 27/30; A23L 2/60; A61K 9/0053; A61K 36/062; A23V 2002/00

(57) ABSTRACT

The present invention generally relates to the use of a sweetening composition comprising (i) a mycelia of an ascomycete or an aqueous extract thereof or (ii) an aqueous extract of a fruiting body of an ascomycete, to provide improved flavor to a product for oral administration, as well as to a sweetening composition comprising (i) a mycelia of an ascomycete or an aqueous extract thereof or (ii) an aqueous extract of a fruiting body of an ascomycete as well as to compositions comprising combinations of sweetening compositions and a product for oral administration.

### CROSS REFERENCE TO RELATED APPLICATIONS

This application is a continuation application of U.S. application Ser. No. 17/421,612, filed Jul. 8, 2021, which applications claims the benefit of and priority to PCT Application No. PCT/US2020/012955, filed Jan. 9, 2020, and U.S. provisional Application Ser. No. 62/790,365, filed Jan. 9, 2019, each of which is incorporated herein by reference in its entirety.

### BACKGROUND OF THE INVENTION

Excess intake of nutritive sweeteners has long been associated with diet-related health issues, such as obesity, heart disease, metabolic disorders and dental problems. Accordingly, consumers are increasingly looking for ways to decrease the amount of nutritive sweeteners in their diets. Manufacturers are responding to this demand by seeking to develop replacements for nutritive Sweeteners that are better able to mimic the desirable taste and functional properties of the nutritive sweeteners.

Zero or low-calorie sweeteners derived from, preferably, natural sources are desired to limit the negative effects of high sugar consumption (e.g., diabetes and obesity, among others.) Commonly known zero or low-calorie sweeteners include aspartame, acesulfame potassium, luo han guo (monk) fruit extract, neotame, saccharin, stevia and sucralose. However, these sweeteners have taste defects such as bitterness.

A truffle is the fruiting body of a subterranean ascomycete fungus including genera which belong to the class Pezizomycetes and the Pezizales order. Truffles are ectomycorrhizal fungi and are therefore usually found in close association with tree roots.

There remains a need in the art to produce new low or zero calorie sweeteners with improved tastes from natural sources. There remains a need in the art to economically produce such sweetening compositions from potential sources of the same from ascomycetes fungal species using aqueous or solid phase production of mycelial inoculum of fungal species. There remains a need for finding a simple, fast and economical process for obtaining mycelium and/or fruiting bodies in pure culture of these species.

### SUMMARY OF THE INVENTION

The present invention generally relates to the use of a sweetening composition comprising (i) a mycelia truffle of family Terfeziaceae or an aqueous extract thereof or (ii) an aqueous extract of a fruiting body of truffle of family Terfeziaceae, to provide improved flavor to a product for oral administration, as well as to a sweetening composition comprising (i) a mycelia of truffle of family Terfeziaceae or an aqueous extract thereof or (ii) an aqueous extract of a fruiting body of truffle of family Terfeziaceae, as well as to compositions comprising combinations of sweetening compositions and a product for oral administration.

### DETAILED DESCRIPTION OF THE INVENTION

The term “product for oral administration” may refer to a comestible product such as a food product, or a beverage product; a medicinal product, or a supplement product such as a herbal supplement. As used herein, the term “medicinal product” includes both solids and liquid compositions which are ingestible non-toxic materials which have medicinal value or comprise medicinally active agents such as cough syrups, cough drops, aspirin and chewable medicinal tablets. An oral hygiene product is also a product for oral administration and includes solids and liquids such as toothpaste or mouthwash.

In general terms, the present invention contemplates that food or beverage products may include a sweetening composition of the invention in an amount of up to about 99% by weight relative to the total weight of the food or beverage product, for example in an amount from about 1% by weight to about 99% by weight. All intermediate weights (i.e., 2%, 3%, 4%, . . . 90%, 95%, 99%) by weight relative to the total weight of the food or beverage products are contemplated, as are all intermediate ranges based on these amounts.

The compositions of the invention may include a “comestibly, biologically or medicinally acceptable carrier or excipient” which can include a solid or liquid medium and/or composition that is used to prepare a desired dosage form of the inventive compound, in order to administer the inventive compound in a dispersed/diluted form, so that the biological effectiveness of the inventive compound is maximized. A comestibly, biologically or medicinally acceptable carrier includes many common food ingredients, such as water at neutral, acidic, or basic pH, fruit or vegetable juices, vinegar, marinades, beer, wine, natural water/fat emulsions such as milk or condensed milk, edible oils and shortenings, fatty acids, low molecular weight oligomers of propylene glycol, glyceryl esters of fatty acids, and dispersions or emulsions of such hydrophobic substances in aqueous media, salts such as sodium chloride, wheat flours, solvents such as ethanol, solid edible diluents such as vegetable powders or flours, or other liquid vehicles, dispersion or suspension aids, surface active agents, isotonic agents; thickening or emulsifying agents, preservatives, solid binders, lubricants and the like.

A “flavor” herein refers to the perception of taste and/or smell in a subject, which include sweet, sour, salty, bitter, umami, and/or savory. The subject may be a human or an animal. The sweetening compositions of the invention as described herein may also optionally include other flavors, such as sour, salty, bitter, umami, and/or savory.

“Sweet flavoring agent,” “sweet compound” or “sweet receptor activating compound” refers to a composition that elicits a detectable sweet flavor in a subject, e.g., sucrose, fructose, glucose, and other known natural saccharide-based sweeteners, or known artificial sweeteners such as saccharine, cyclamate, aspartame, and the like as is further discussed herein, or a material that activates a T1R2/T1R3 receptor in vitro. The subject may be a human or an animal.

A sweet flavoring agent or sweetening composition may be used in an effective amount, which refers to an amount of a sweetening composition of the invention that is sufficient to induce sweet taste in a subject when present in a product for oral administration.

Food or beverage products that may be contemplated in the context of the present invention include baked goods; sweet bakery products, (including, but not limited to, rolls, cakes, pies, pastries, and cookies); pre-made sweet bakery mixes for preparing sweet bakery products; pie fillings and other sweet fillings (including, but not limited to, fruit pie fillings and nut pic fillings such as pecan pie filling, as well as fillings for cookies, cakes, pastries, confectionary products and the like, such as fat-based cream fillings); desserts, gelatins and puddings; frozen desserts (including, but not limited to, frozen dairy desserts such as ice cream—including regular ice cream, soft serve ice cream and all other types of ice cream—and frozen non-dairy desserts such as non-dairy ice cream, sorbet and the like); carbonated beverages (including, but not limited to, soft carbonated beverages); non-carbonated beverages (including, but not limited to, soft non-carbonated beverages such as flavored waters and sweet tea or coffee based beverages); beverage concentrates (including, but not limited to, liquid concentrates and syrups as well as non-liquid concentrates, such as freeze-dried and/or powder preparations); yogurts (including, but not limited to, full fat, reduced fat and fat-free dairy yogurts, as well non-dairy and lactose-free yogurts and frozen equivalents of all of these); snack bars (including, but not limited to, cereal, nut, seed and/or fruit bars); bread products (including, but not limited to, leavened and unleavened breads, yeasted and un-yeasted breads such as soda breads, breads comprising any type of wheat flour, breads comprising any type of non-wheat flour (such as potato, rice and rye flours), gluten-free breads); pre-made bread mixes for preparing bread products; sauces, syrups and dressings; sweet spreads (including, but not limited to, jellies, jams, butters, nut spreads and other spreadable preserves, conserves and the like); confectionary products (including, but not limited to, jelly candies, soft candies, hard candies, chocolates and gums); sweetened breakfast cereals (including, but not limited to, extruded (kix type) breakfast cereals, flaked breakfast cereals and puffed breakfast cereals); and cereal coating compositions for use in preparing sweetened breakfast cereals. Other types of food and beverage product not mentioned here but which conventionally include one or more nutritive sweetener may also be contemplated in the context of the present invention.

As a consequence of the complete or partial replacement of nutritive sweeteners in the food or beverage products of the present invention, the food or beverage products of the present invention may be useful as low calorie or dietetic products, medical foods/products (including pills and tablets), and sports nutrition products, and may be particularly suitable for food or beverage products requiring a lower sweetness at a given soluble solids level.

In some embodiments, the sweetening composition of the invention can be supplemented with other nutritional or non-nutritional sweeteners to form a sweetener system. The sweetener system may comprise the sweetening composition of the invention, a bulking agent such as maltodextrose, gum acacia and the like, and at least one high intensity sweetener. The composition may be provided as liquid composition or a dried blend.

In an embodiment, the present invention includes a process for enhancing the sweet taste of a product for oral administration, comprising the addition of a sweetening composition of the invention. In an embodiment, a sweetening composition of the invention can comprise, consist of, or consist essentially of (i) a mycelia of truffle of family Terfeziaceae, in embodiments, a truffle of genus Terfezia, Tirmania, or Mattirolomyces; or an extract thereof or (ii) an extract of a fruiting body of truffle of family Terfeziaceae to the food or beverage. In one embodiment, the fungus comprises, consists of, or consists essentially of genus Mattirolomyces, for example, comprising, consisting of, or consisting essentially of Mattirolomyces terfezoides or Mattirolomyces (E. Fisch) (Pezizaceae).

“Mycelia” refers to a biomass which is the result of a culturing step as described herein and has been harvested. After harvest, cultures can be processed according to a variety of methods. In one embodiment, the mycelia is pasteurized or sterilized. In one embodiment, the mycelia is dried according to methods as known in the art. Additionally, concentrates and isolates of the material may be prepared using variety of solvents or other processing techniques known in the art. In one embodiment the material is pasteurized or sterilized, dried and powdered by methods known in the art. Drying can be done in a desiccator, vacuum dryer, conical dryer, spray dryer, fluid bed or any method known in the art. For storage, the mycelia may be dried, or alternatively, can be stored at 4° C., or alternatively frozen. The dried, frozen or fresh mycelia can be optionally blended, pestle, milled or pulverized, or other methods as known in the art prior to use.

In one embodiment, the truffle (fruiting body) or a mycelia are extracted using an aqueous extraction technique. Such aqueous extraction techniques are known in the art. Generally, in a first step, the truffle or mycelia are diced, ground, or macerated. This step may be performed on a frozen, cold, room temperature, or heated sample. In some embodiments, a maceration step under liquid nitrogen is performed.

Following the grinding or maceration step, an aqueous solution is added. In one embodiment, the aqueous solution is water. The water may be buffered in some embodiments with food-grade buffers to attain a specific pH, such as, for example, pH 7, as known in the art. In embodiments, the range of pH for extraction can vary from about pH 5 through pH 8. Optionally the water may contain food-grade salts. Relative amounts of aqueous solution to add to a truffle or mycelia can include from 1:1 v/w (volume aqueous solution to wet weight of truffle or mycelia) to 50:1 w/v, although commonly, ranges of 2:1 v/w to 10:1 v/w are used.

In embodiments, the aqueous solution can be cooled (e.g., 4° C., or between 4° C. and about 10° C.), room temperature (about 22° C.), or heated (about 40° C., about 50° C., about 60° C., about 70° C., about 80° C., about 90° C., or about 95° C. or 100° C.; or superheated under pressure to about 125° C.). In one embodiment, the extraction is carried out with 4° C. water for about 30 minutes.

The non-extracted material may then be separated from the extracted material by, e.g., any separation technique known in the art, such as filtration or centrifugation, for example, low speed centrifugation. The present inventors found that the sweet taste that is found in the truffle and in the mycelia, appears in the aqueous extract.

The extract may be optionally heated. The sweet taste is found to be preserved in a heating step. An optional pre-extraction step may be performed using a nonpolar solvent such as hexane or a polar solvent such as an alcohol to remove nonpolar components.

Optionally, the truffle (fruiting body) or a sample of mycelia are extracted using an aqueous alcoholic extraction technique as known in the art.

Optionally, the extract may be further processed to “clarify” the flavor. In one embodiment, an aqueous extract as described herein may be bound to an anion exchange column at low pH, and then eluted; or the aqueous extract as described herein may be separated via size exclusion chromatography to separate a clarified sweet flavor from a fungal flavor.

In an embodiment, the present invention includes a composition comprising, consisting essentially of, or consisting of a combination of a product for oral administration and a sweetening composition comprising (i) a mycelia of truffle of family Terfeziaceae or an extract thereof or (ii) an extract of a fruiting body of truffle of family Terfeziaceae. The present invention also includes a sweetening composition comprising, consisting essentially of, or consisting of an extract of truffle of family Terfeziaceae or extract of truffle of family Terfeziaceae body. In one embodiment, the fungus comprises, consists of, or consists essentially of genus Mattirolomyces, for example, comprising, consisting of, or consisting essentially of Mattirolomyces terfezoides or Mattirolomyces (E. Fisch) (Pezizaceae).

In one embodiment of the present invention, the sweetening composition comprises mycelia, wherein the mycelia of the invention is obtained by a process comprising the steps of: inoculating an aqueous medium with an ascomycete fungus culture; culturing the aqueous medium and the ascomycete fungus culture in submerged culture to obtain a mycelium and/or a fruiting body, wherein the ascomycete fungus culture comprises truffle of family Terfeziaceae; and collecting the mycelia. In one embodiment, the fungus comprises, consists of, or consists essentially of genus Mattirolomyces, for example, comprising, consisting of, or consisting essentially of Mattirolomyces terfezoides or Mattirolomyces (E. Fisch) (Pezizaceae).

The aqueous media is primarily intended as a growth media for the fungi in an aqueous liquid culture, optionally, submerged liquid culture. Optionally, the culture is agitated during growth. Therefore, any art-known media which is capable of supporting growth of a ascomycete fungi to the desired levels of growth can be used with the present invention. Art known media for growth of fungi in liquid culture can be defined or undefined and generally will include a carbon source, a nitrogen source, and optionally, additional components to support growth.

As used herein, the terms “culturing,” “myceliation,” and “fermentation,” are used interchangeably. All these terms refer to a process of bulk growth or maintenance of microorganisms, which can be single celled or multicellular, including, without limitation, the fungi referred to herein, on a medium. Growth or maintenance can refer to organisms in all growth phases, e.g., lag phase, log phase, or stationary phase.

In an embodiment, the aqueous media can include a general nutritional media for the growth of filamentous fungi, and in embodiments includes a carbon source, a nitrogen source, vitamins and/or nutritional salts. Nitrogen sources can optionally comprise, consist of, or consist essentially of, in addition to the protein sources identified elsewhere herein for e.g., the second aqueous media, and/or nitrogen sources such as peptone, yeast extract, malt extract, amino acids, ammonium or nitrate compounds. Carbon sources can comprise, consist of, or consist essentially of, without limitation, glucose (dextrose), molasses, maltodextrose, fructose, mannose and/or sucrose, to name a few commonly-used carbon sources. Salts can include potassium, iron, magnesium, zinc, and manganese.

The ascomycete fungal culture can comprise, consist of, or consist essentially of an organism from subterranean ascomycete fungus (truffle fungus), including species from Tuber, many other genera of fungi are classified as truffles including Geopora, Peziza, Choiromyces, Leucangium, and others. The fungal species may belong to the class Pezizomycetes and the Pezizales order and family Terfeziaceae, and fungal species may include a species from genera Terfezia and Tirmania (“desert truffles” of Africa and the Middle East) and the genera Mattirolomyces. In one embodiment, the fungus comprises, consists of, or consists essentially of genus Mattirolomyces, for example, comprising, consisting of, or consisting essentially of Mattirolomyces terfezoides or Mattirolomyces (E. Fisch) (Pezizaceae). Mattirolomyces terfezioides truffle (Hungarian sweet truffle). Mattirolomyces terfezioides (Mattir.) E. Fisch., the type species of Mattirolomyces E. Fisch. (Pezizaceac, Pezizales), was originally described from Northern Italy by Mattirolo (1887) in the genus Choiromyces Vittad. Fischer (1938) erected a monotypic genus Mattirolomyces using Choiromyces terfezioides Mattir. as the type. Molecular phylogenetic analyses supported Mattirolomyces to be a separate genus from Terfezia within the same family, Pezizaceae, thus making the name M. terfezioides fixed. Unlike Terfezia species (desert truffle), which are mostly found in arid to semi-arid sandy environments in Mediterranean region and form mycorrhizae with herbaceous species of Cistaceae (Díez et al., 2002), M. terfezioides is often found under artificially planted trees [e.g. Robinia pseudoacacia L., Diospyros kaki Thunb. and Prunus avium (L.) L.] in southern and central Europe. The typical characters of M. terfezioides include the whitish to yellowish brown ascomata with subsolid whitish to yellowish gleba with minute pockets asci and globose ascospores with blunt spines connected in an irregular alveolate reticulum 1e⁴ (e⁵) mm high.

Mattirolomyces terfezioides grows mainly on the sandy soils deposited by the Danube, slightly alkaline to neutral ones, high in humus, and that is why it is also called “sand truffle”. This sweet truffle grows in semi-humid climates, although it is included within the “desert truffles”. Although M. terfezioides is a truffle that comes to the surface when ripens, when they are detected by dogs before cracking the surface of the ground, they are of better quality. Mattirolomyces terfezioides are regular globose shaped with the size between 1-20 cm. Peridium very thin, white color at first, then becoming ochre, when ripens. The gleba is ochre with white veins. They have a softer and more watery consistency than Terfezias, going off earlier.

Truffles (fruiting body) of the named species herein may be obtained in situ in their natural environments, or may be obtained commercially in season in markets. Mycelia from truffles of the named species herein may be subcultured from the truffles using techniques known in the art, or, alternatively, may be obtained commercially, for example, from suppliers of fungal cultures. Suppliers include American Type Culture Collection (ATCC), Virginia, USA, Agricultural Research Service Culture Collection (NRRL), Banque Européenne des Glomales (BEG), Belgian Co-ordinated Collections of Micro-organisms (BCCM), Canadian Collection of Fungal Cultures (CCFC), Centraalbureau voor Schimmelcultures (CBS), Czech Collection of Fungi (CCF), Fungal Genetics Stock Center (FGSC), among others. In one embodiment, cultures of Mattirolomyces terfezioides are obtained from the Westerdijk Fungal Biodiversity Institute, Netherlands. Strains are typically received as “master culture” PDY slants in 50 mL test tubes. For plating, small pieces of culture are typically transferred into sterile shake flasks (e.g. 250 mL) so as not to contaminate the flask filled with a sterilized media (liquid media recipes are discussed below). Inoculated flasks shake for approximately ten hours and aliquots of said flasks are then plated onto prepared Petri plates of a sterile agar media. One flask can be used to prepare dozens to potentially hundreds of Petri plate cultures. Cultures received as truffles can be carefully washed with dilute bleach solutions and cut into pieces and separately plated using sterile procedure.

In one embodiment, the aqueous media further comprises, consists of, or consists essentially of additional excipients as defined herein. Excipients can comprise any other components known in the art to potentiate and/or support fungal growth, and can include, for example, nutrients, such as proteins/peptides, amino acids as known in the art and extracts, such as malt extracts, meat broths, peptones, yeast extracts and the like; energy sources known in the art, or carbon sources, such as carbohydrates including glucose and sucrose; essential metals and minerals as known in the art, which includes, for example, calcium, magnesium, iron, trace metals, phosphates, sulphates; buffering agents as known in the art, such as phosphates, acetates, and optionally pH indicators (phenol red, for example). Excipients may include carbohydrates and/or sources of carbohydrates added to media at 5 to10 g/L. It is usual to add pH indicators to such formulations.

Excipients may also include peptones/proteins/peptides, as is known in the art. These are usually added as a mixture of protein hydrolysate (peptone) and meat infusion, however, as used in the art, these ingredients are typically included at levels that result in much lower levels of protein in the media than is disclosed herein. Many media have, for example, between 1% and 5% peptone content, and between 0.1 and 5% yeast extract and the like.

In one embodiment, excipients include for example, yeast extract, malt extract, maltodextrin, peptones, and salts such as diammonium phosphate and magnesium sulfate, as well as other defined and undefined components such as potato or carrot powder. In some embodiments, organic (as determined according to the specification put forth by the National Organic Program as penned by the USDA) forms of these components may be used.

In one embodiment, a media suitable for the growth of yeast, molds or filamentous fungi suitable for the present invention includes Malt Yeast Peptone Glucose (MYPG) media, as known in the art and disclosed herein; a suitable media also includes MMN (also disclosed herein). Relative amounts of each component in the media may be adjusted as suitable as determined by one of skill in the art. Excipients may also optionally comprise, consist of, or consist essentially of citric acid and an anti-foam component. The anti-foam component can any anti-foam component known in the art, such as a food-grade silicone anti-foam emulsion or an organic polymer anti-foam (such as a polypropylene-based polyether composition).

At the end of the culturing period, the sweetening composition may be collected. The collected material which is the sweetening composition may include the entire contents of the fermentation vessel. Alternatively, the sweetening composition may include only the solid contents of the fermentation, optionally including the biomass, which may be collected by art known methods, such as centrifugation or filtration.

The method may optionally include a heat-treatment and/or concentrating step. Such treatments include, without limitation, heating the sweetening composition by heating by any method known in the art. Suitable heating means can be selected from conventional means and optionally include a drum dryer, a flash dryer, a hot plate, an extruder/heater, a concentrator, and other such conventionally known techniques. The heat treatment can be carried out at normal pressures or under increased pressures.

Before, during, or after the heating step, the sweetening composition may be concentrated (e.g., to dewater the material), by methods known in the art. In embodiments, the heating and concentration step may be carried out concurrently. In embodiments, the remaining moisture after the heating step may be 30% by weight or less, 25% by weight or less, 20% by weight or less, 15% by weight or less, or 10% by weight or less; or between 20 and 30% by weight, or between 10 and 20% by weight or less. In other embodiments, the volume of the sweetening composition can be reduced about 50%, about 70%, about 80%, about 90% or about 95%.

Optionally, or alternatively, the sweetening composition may be spray-dried by known techniques to form a low-moisture product.

In an embodiment, the sweetening composition is capable of modulating sweet perception by human sensory testing. It is to be understood that the methods of the invention only optionally include a step of determining whether the sweetening composition differs from a control material. Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine sweetening can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone.

In the instant invention, the sweetening capacity of the sweetening composition was measured by a particular method as described herein; however, it should be understood that there are any number of ways one of skill in the art could measure the sensory differences. For the purposes of clarity, the present invention's method for quantitating the sweetening potential and overall sensory perception of different methods is described. However, an appropriate method which differs from the one proposed may be used by one of skill in the art.

The method may further include wherein at least one culturing step can include a fruiting induction step. Fruiting induction can include subjecting the culture to methods known in the art to induce fruiting in filamentous fungi in either solid or aqueous media, and can include nutrient deprivation, osmotic shock, pH shock, thermal shock, light treatment, or exposure to a fruiting induction compound.

As mushroom formation is closely tied to nutrient depravation, the literature shows that complete fungal colonization of a substrate accompanied with concurrent nutrient depravation is required for induction a full fruiting. The conserved signaling molecule cAMP is known to be produced as a signal molecule under low ATP conditions and induces a nutrient deprivation alarm response in bacteria, and a fruiting response in the cellular slime mold Dictyostelium discoideum. This potent activator also signals mycelium to activate fruition pathways, as cAMP pathway involvement in fruition is known to be present in at least one member of the Basidiomycetes, Schizophyllum commune.

Methods known in the art to induce fruiting in solid culture include low levels of CO₂, nutrient deprivation, osmotic shock, pH shock, thermal shock, light treatment, or exposure to a fruiting induction compound such as veratryl alcohol, shiikimic acid, and/or glycine.

In general, mycelia can be produced using a number of different methods known in the art. In one embodiment, a subculture of mycelia may be developed from a truffle or fruiting body using methods known in the art relying on ectomycorrhiza present in the truffle to develop the mycelia in culture. Alternatively, mycelia may be obtained from commercial sources.

The first aqueous media may include a sterilized liquid nutrient media. To start this process, a pure culture of mycelium is propagated in small containers which are then used to inoculate a larger volume of liquid in production scale tanks. The process takes place under aerobic conditions that are maintained by mechanically stirring the liquid and pumping sterile fresh air into the tank. Metabolic gases such as CO₂ are allowed to escape. By maintaining a consistent temperature, mycelia grow and expand into a true biomass. After 3-8 days, the mycelial biomass is separated from the fluid media, dried and ground to a powder. The fluid can also be purified to harvest any extracellular compounds that the mycelia may have produced.

The method may also comprise the optional step of sterilizing the aqueous media prior to inoculation by methods known in the art, including steam sterilization and all other known methods to allow for sterile procedure to be followed throughout the inoculation and culturing steps to enable culturing and myceliation by pure fungal strains. Alternatively, the components of the media may be separately sterilized, and the media may be prepared according to sterile procedure.

In one embodiment, the mycelia of the invention are cultivated in a media that has a pH adjusted to greater than pH 6, greater than pH 6.5, greater than pH 7, or greater than pH 7.2 for optimal growth. Appropriate media include MMN or MYPG (disclosed herein).

In one embodiment, maintaining and propagating mycelia for use for inoculating the aqueous media as disclosed in the present invention may be carried out as follows. For example, a propagation scheme that can be used to continuously produce material according to the methods is discussed herein. Once inoculated with master culture and subsequently colonized, Petri plate cultures can be used at any point to propagate mycelium into prepared liquid media. As such, plates can be propagated at any point during log phase or stationary phase.

The culturing step of the present invention may be performed by methods (such as sterile procedure) known in the art and disclosed herein and may be carried out in a fermenter, shake flask, bioreactor, or other methods. In a shake flask, in one embodiment, the agitation rate is 50 to 240 RPM, or 85 to 95 RPM, and incubated for 1 to 90 days. In another embodiment the incubation temperature is 21 to 35° C. In another embodiment the incubation temperature is 30-33° C. Liquid-state fermentation agitation and swirling techniques as known in the art are also employed which include mechanical shearing using magnetic stir bars, stainless steel impellers, injection of sterile high-pressure air, the use of shaker tables and other methods such as lighting regimen, batch feeding or chemostatic culturing, as known in the art.

In one embodiment, culturing step is carried out in a bioreactor which is ideally constructed with a torispherical dome, cylindrical body, and spherical cap base, jacketed about the body, equipped with a magnetic drive mixer, and ports to provide access for equipment comprising DO, pH, temperature, level and conductivity meters as is known in the art. Any vessel capable of executing the methods of the present invention may be used. In another embodiment the set-up provides 0.1-5.0 ACH. Other engineering schemes known to those skilled in the art may also be used.

The reactor can be outfitted to be filled with water. The water supply system is ideally water for injection (WFI) system, with a sterilizable line between the still and the reactor, though RO or any potable water source may be used so long as the water is sterile. In one embodiment the entire media is sterilized in situ while in another embodiment concentrated media is sterilized and diluted into a vessel filled water that was filter and/or heat sterilized, or sufficiently treated so that it doesn't encourage contamination over the colonizing fungus. In another embodiment, high temperature high pressure sterilizations are fast enough to be not detrimental to the media. In one embodiment the entire media is sterilized in continuous mode by applying high temperature between 130° and 150° C. for a residence time of 1 to 15 minutes. Once prepared with a working volume of sterile media, the tank can be mildly agitated and inoculated. Either as a concentrate or whole media volume in situ, the media can be heat sterilized by steaming either the jacket, chamber or both while the media is optionally agitated. The medium may optionally be pasteurized instead.

A typical process would pass a culture from master culture, to Petri plates, to flasks, to seed bioreactors to the final main bioreactor when scaling the method of the present invention. To reach large volumes, 3-4 seeds may be used. The media of the seed can be the same or different as the media in the main.

In another embodiment, the present invention includes a method to prepare a sweetening composition which includes inoculating a solid-state medium with an ascomycete fungus culture. The method further optionally includes culturing the solid state medium and the ascomycete fungus culture to obtain a mycelium and/or a fruiting body, wherein the ascomycete fungus culture comprises a truffle of family Terfeziaceae. The sweetening composition may include on or more of the solid-state medium, and/or the cultured mycelium and/or the cultured fruiting body. The method may further include a fruiting induction step as described herein.

Appropriate solid-state media can include any known in the art suitable for solid state fermentation (SSF). To identify the most suitable type of inoculum to be employed in SSF, the nature of fungi involved has to be taken into consideration. The commonly applied inoculum preparation methods for SSF include spore suspension, mycelia disc, mycelia suspension and pre-inoculated substrates. In some embodiments, inoculation by mycelia disc or mycelia suspension is used. Inoculum in the form of mycelia disc is prepared by cutting the agar plug from the periphery of the actively grown fungi. The mycelia disc can be directly used to inoculate the substrate. Mycelia suspension is also a choice of inoculum in SSF. First, the mycelia mat or mycelia disc from an agar plate with actively grown fungi needs to be transferred into a liquid medium before incubating it. After the incubation period, washing and homogenization of the fungal pellets is performed. Also, a pre-inoculated substrate may be used as the inoculum for SSF. In general, this type of inoculum was prepared by transferring the mycelia disc onto the cooked or autoclaved wheat grains. It is then incubated at room temperature for a period of time ranges from 6 to 21 days. Calcium carbonate can be added to the solid phase medium before inoculation to adjust the pH into a range which is suitable for a particular fungus to grow.

Process conditions in SSF, such as the composition of fermentation medium, fermentation duration, pH, temperature and moisture content of the substrate can affect growth. Sample substrates include soybean hull, corn fiber, corn stover, rice straw, wheat flour, sage waste, tree leaves, beech leaves, wheat straw, tree sawdust, rice bran, reed grass, and excipients can include malt extract, urea, ammonium salts, phosphate salts, potassium salts, calcium salts, sodium salts, magnesium salts, peptones, yeast extract, manganese salts, copper salts, iron salts, zinc salts, and the like, as known in the art.

Determining when to end the culturing step and to harvest the sweetening composition, having acceptable taste, flavor and/or aroma profiles, can be determined in accordance with any one of a number of factors as defined herein, such as, for example, visual inspection of mycelia, microscope inspection of mycelia, pH changes, changes in dissolved oxygen content, changes in protein content, amount of biomass produced, and/or assessment of taste profile, flavor profile, or aroma profile. In one embodiment, harvest can be determined by tracking protein content during culturing and harvest before significant catabolism of protein occurs. The present inventors found that protein catabolism can initiate in bioreactors at 30-50 hours of culturing under conditions defined herein. In another embodiment, production of a certain amount of biomass may be the criteria used for harvest. For example, biomass may be measured by filtering, such through a filter of 10-1000 μm, and has a protein concentration between 0.1 and 25 g/L; or in one embodiment, about 0.2 to 0.4 g/L. In one embodiment, harvest can occur when the dissolved oxygen reaches about 10% to about 90% dissolved oxygen, or less than about 80% of the starting dissolved oxygen. Additionally, mycelial products may be measured as a proxy for mycelial growth, such as, total reducing sugars (usually a 40-95% reduction), β-glucan and/or chitin formation; harvest is indicated at 10² to 10⁴ ppm. Other indicators include small molecule metabolite production depending on the strain or nitrogen utilization (monitoring through the use of any nitrogenous salts or protein, cultures may be stopped just as protein starts to get utilized or may continue to culture to enhance the presence of mycelial metabolites).

In embodiments, the present invention includes sweetening composition made by the methods of the instant invention. In other embodiments, the present invention includes a mycelia and/or fruiting body of an ascomycete fungal culture, e.g., truffle of family Terfeziaceae grown in liquid culture. The present invention also includes the use of a sweetening composition comprising (i) a mycelia of truffle of family Terfeziaceae or an extract thereof or (ii) an extract of a fruiting body of truffle of family Terfeziaceae to provide improved flavor to a product for oral administration. In one embodiment, the fungus comprises, consists of, or consists essentially of genus Mattirolomyces, for example, comprising, consisting of, or consisting essentially of Mattirolomyces terfezoides or Mattirolomyces (E. Fisch) (Pezizaceae).

In another embodiment, the methods of the invention include a method for improving the sweet flavor of a product for oral administration, comprising adding to the product for oral administration a sweetening composition made by the methods of the invention. Amounts to add can be determined by methods known in the art, e.g., using sensory testing as a guide.

The following examples further illustrate the invention but, of course, should not be construed as in any way limiting its scope.

### EXAMPLES

### Example 1

A medium containing 10 g/L maltose, 1 g/L pea protein and 1 g/L yeast extract is formulated in a 1 L volumetric flask and 200 mL is dispensed into 5, 1 L Erlenmeyer flasks and sterilized in an autoclave. Once the media is cool, each flask is inoculated with ⅛ of a 90 mm Petri plate fully colonized with Mattirolomyces terfezioides. Before inoculation, each flask is plated onto Petri film to ensure for sterility. The flasks are incubated at 25° C. and shaken at 120 RPM on a shaker table. One flask is not inoculated to serve as a control. After 10 days, the contents of every flask are pasteurized and tasted. It is found the inoculated flasks are much more intensely sweet than the control.

### Example 2

A polypropylene bag with 0.2 μm breather patch (known as an autoclave bag) is filled with 400 g of a wild rice mix and has 200 mL RO water added to it and is sterilized. Once cool, the bag is inoculated with 40 mL of a liquid medium as prepared in Example 1 (10 days culture with Mattirolomyces terfezioides). After 1 month the bag is fully colonized with Mattirolomyces terfezioides and is pasteurized. The grain is tasted and is found to be intensely sweet.

### Example 3

A medium containing 10 g/L maltose, 1 g/L pea protein and 1 g/L yeast extract is formulated in a 1 L volumetric flask and 200 mL is dispensed into 5, 1 L Erlenmeyer flasks and sterilized in an autoclave. Once the media is cool, each flask is inoculated with ⅛ of a 90 mm Petri plate fully colonized with Mattirolomyces terfezioides. Before inoculation, each flask is plated onto Petri film to ensure for sterility. The flasks are incubated at 25° C. and are shaken at 120 RPM on a shaker table. One flask is not inoculated to serve as a control. After 10 days, a 2 mL aliquot of sterilized Agaricus bisporus fruit body extract is added to the culture (to induce fruit body genetic pathway expression). After another 4 days the contents of every flask are pasteurized and are tasted. It is found the flasks that had been inoculated are much more intensely sweet than the control.

### Example 4. Growth of Mycelia

Mattirolomyces terfezioides culture was received from a commercial source and the phylogenetic identity of the culture was confirmed by ITS (internal transcribed spacers) analysis (data not shown). The samples were received on agar slants and plated on MYPG with antibiotic selection, and subcultured to seed into liquid MYPG media. A clump was finely chopped with a sterile blade and was used for each of these experiments. The sample was divided and cultured in several types of media. All samples were incubated at 26° C. for up to 40 days. It was found that best growth is achieved at higher pH, 7.0 and above. Table 1 shows the media conditions used, with growth observed with each media evaluated from + to +++++, with more being better growth.

TABLE 1

| Media | Growth |
|---|---|
| MMN_Glucose | +++++ |
| MMN_Sucrose | +++++ |
| MMN_Glucose + IAA 100 μM | + |
| MMN_Glucose + IAA 500 μM | + |
| LT_Glucose + uracil − glucaronic + glutamic | ++ |
| LT_Glucose + uracil − glucaronic − glutamic + nitrate | ++ |
| LT_Sucrose + uracil − glucaronic + glutamic | ++ |
| LT_Sucrose + uracil − glucaronic − glutamic + nitrate | ++ |
| MYPG Buffered_Glucose | +++ |
| MYPG Buffered_Sucrose | +++ |

TABLE 2
Table 2. MMN media.

| | Amount | |
|---|---|---|
| | 1000 mL | |
| Component | (g/L) | |
| Malt Extract | 3.0 | g |
| Sucrose (or Glucose) | 10.0 | g |
| Glucose (or Sucrose) | 10.0 | g |
| (NH₄)₂HPO₄ | 0.25 | g |
| KH₂PO₄ | 0.50 | g |
| MgSO₄•7H₂O | 0.15 | g |
| CaCl₂•2H₂O | 0.067 | g |
| FeCl₃ (1% solution) | 1.0 ml (1 mg/L) | |
| NaCl | 0.025 | g |
| Thiamine•HCl | 0.1 | mg |

| | |
|---|---|
| dH₂O | q.s. to 1 L |

TABLE 3
Table 3. LT medium

| Component | Component | |
|---|---|---|
| YNB: Yeast Nitrogen Base w/o AA, w/o AS | 1.7 | g |
| DOM: DropOut Mix AA (-URA) | 2.0 | g |
| Glucose | 10.0 | g |
| Sucrose | 10.0 | g |
| Potassium phosphate Monobasic | 2.0 | g |
| Magnesium Sulfate | 2.0 | g |
| Uracil | 76.0 | mg |
| L-Glutamic acid (or ammonium nitrate) | 2.5 | g |
| Ammonium Nitrate (or L-Glutamic acid) | 1.5 | g |

| | |
|---|---|
| dH₂O | q.s. to 1 L |

Adjust to pH 7.0.

TABLE 4
Table 4. MYPG-Liquid

| | Final |
|---|---|
| Compound | Concentration |
| Malt Extract | 1.0% |
| Yeast Extract | 0.4% |
| Peptone | 0.1% |
| Potassium Phosphate Monobasic | 0.2% |
| D-Glucose (or Sucrose) | 0.4% |
| Sucrose (or Glucose) | 0.4% |

Results: Results show that best growth was achieved with MMN media, followed by MYPG media.

Mycelia grown by these techniques were found to have a sweet taste, similar to the taste characteristics described in Example 5, using preparation techniques shown in Example 5.

### Example 5

Fresh Mattirolomyces terfezioides truffles were obtained in situ using appropriate procedures and permissions in their natural range. Fresh samples (29 in total) were shipped to MycoTechnology, Inc. facilities and were gently washed in RO water, then frozen in liquid nitrogen and stored at −80° C. The average moisture content of the truffles was 83.6% plus or minus 4.6%.

Aqueous extraction of the truffles was performed as follows. Eight different samples of truffle were pestled in liquid nitrogen to grind into a powder, then 5:1 v/w truffle of 4° C. water was added and allowed to incubate at 30 minutes at 4° C. The extracted material was then subjected to low-speed brief centrifugation and the filtrate was tasted “neat.” The sweetness intensity was rated between 0 for no sweetness and 10 for extremely sweet. Of the 8 samples, the sweetness was rated as follows:

TABLE 5
Table 5. Sweetness of various truffle samples.

| sample | Sweetness intensity | Notes |
|---|---|---|
| 1 | 5 | Sweet taste at end |
| 2 | 8 | Sweet taste is upfront and |
| | | intensifies at mid-end and lingers |
| 3 | 7 | Sweetness is upfront and |
| | | intensifies at mid-end and lingers. |
| 4 | 5 | Sweet upfront, low sweet linger |
| 5 | 4 | Sweetness less strong |
| 6 | 3 | Low sweetness |
| 7 | 6 | A mild and clean sweet taste |

The aqueous extracts had the following sweetness upon dilution.

TABLE 6
Table 6. Dilution

| Relative concentration | Overall sweet taste | Lingering sweet taste |
|---|---|---|
| 1 | 9 | 7 |
| ½ | 6 | 5 |
| ¼ | 4 | 4 |
| ⅛ | 3 | 2 |
| 1/16 | 2 | 1 |
| 1/32 | 0 | 1 |

Results: These results showed that the sweet taste in the extracts persisted upon dilution in a dose-dependent fashion.

### Example 6. Heat Tolerance of Aqueous Extract

M. terfezioides aqueous extract prepared as in Example 5 was exposed to either 22° C., 37° C., 50° C., 93° C., or 125° C. (pressurized) for 30 minutes and tasted. See Table 7.

TABLE 7

| | | Sweet perception | Sweet perception- |
|---|---|---|---|
| Temp (° C.) | time (minutes) | initial | lingering |
| 22 | 30 | 10 | |
| 37 | 30 | 7.5 | |
| 50 | 30 | 7.5 | |
| 93 | 0 | 7 | 8 |
| 93 | 2 | 7 | 7.5 |
| 93 | 3 | 5 | 6.5 |
| 93 | 4 | 4.5 | 6.5 |
| 93 | 5 | 4 | 6 |
| 125 | 30 | 2 | |

Results showed that the sweet taste can be heat-treated, although loss of activity is noted with higher temperatures and longer heating time.

Aqueous extract was stored at 4° C. at pH 7 and pH 2 in sodium phosphate buffer, and little to no change in sweetness was observed over an 8 day period.

### Example 7

An aqueous extract prepared as in Example 5 (using 10 mM sodium phosphate pH 6.5) was loaded onto a prepared size exclusion column SEPHACRYL 400-HR in an SPE format (obtained from Waters, Milford, MA), equilibrated to 10 mM sodium phosphate pH 6.5. After the void volume, in the first volume obtained from the column a fungal tasting component was eluted; in the second volume obtained from the column, a sweet tasting component was eluted.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Use of a sweetening composition comprising (i) a mycelia of a truffle of family Terfeziaceae or an aqueous extract thereof or (ii) an aqueous extract of a fruiting body of a truffle of family Terfeziaceae, to provide improved flavor to a product for oral administration.

2. A composition comprising a combination of a product for oral administration and a sweetening composition comprising (i) a mycelia of a truffle of family Terfeziaceae or an aqueous extract thereof or (ii) an aqueous extract of a fruiting body of a truffle of family Terfeziaceae.

3. A composition comprising an aqueous extract of a truffle of family Terfeziaceae a truffle of family Terfeziaceae sweet truffle (Mattirolomyces terfezioides) fruiting body.

4. The use of claim 1, wherein the sweetening composition comprises mycelia or an aqueous extract of mycelia, wherein the mycelia is obtained by a process comprising the steps of:
inoculating an aqueous medium with an ascomycete fungus culture;
culturing the aqueous medium and the ascomycete fungus culture in submerged culture to obtain a mycelium and/or a fruiting body, wherein the ascomycete fungus culture comprises a truffle of family Terfeziaceae; and
collecting the mycelia.

5. The use of claim 4, wherein the aqueous media comprises malt extract, a carbon source, a potassium salt, a magnesium salt, an iron salt, thiamine, and sodium chloride, and has a pH at or above 7.0.

6. The use of claim 4, wherein the ascomycete fungal culture is a submerged liquid fungal culture.

7. The use of claim 4, wherein the step of collecting comprises filtering the cultured mycelium to remove the aqueous media.

8. The use of claim 1, wherein the product for oral administration is a food or beverage product or a medicinal product.

9. The use of claim 8, wherein the product for oral administration is a food product selected from the group consisting of baked goods; sweet bakery products, pre-made sweet bakery mixes for preparing sweet bakery products; pie fillings and other sweet fillings, gelatins and puddings; frozen desserts; yogurts; snack bars; bread products; pre-made bread mixes for preparing bread products; sauces, syrups and dressings; sweet spreads; confectionary products; and sweetened breakfast cereals.

10. The use of claim 8, wherein the product for oral administration is a beverage product selected from the group consisting of carbonated beverages; non-carbonated beverages; and
beverage concentrates.

11. The use of claim 1, wherein the truffle of family Terfeziaceae comprises a truffle of genus Terfezia, Tirmania, or Mattirolomyces.

12. The composition of claim 2 or 3, wherein the sweetening composition comprises mycelia or an aqueous extract of mycelia, wherein the mycelia is obtained by a process comprising the steps of:
inoculating an aqueous medium with an ascomycete fungus culture;
culturing the aqueous medium and the ascomycete fungus culture in submerged culture to obtain a mycelium and/or a fruiting body, wherein the ascomycete fungus culture comprises a truffle of family Terfeziaceae and
collecting the mycelia.

13. The composition of claim 2 or 3, wherein the aqueous media comprises malt extract, a carbon source, a potassium salt, a magnesium salt, an iron salt, thiamine, and sodium chloride, and has a pH at or above 7.0.

14. The composition of claim 2 or 3, wherein the ascomycete fungal culture is a submerged liquid fungal culture.

15. The composition of claim 2 or 3, wherein the step of collecting comprises filtering the cultured mycelium to remove the aqueous media.

16. The composition of claim 2 or 3, wherein the product for oral administration is a food or beverage product or a medicinal product.

17. The composition of claim 16, wherein the product for oral administration is a food product selected from the group consisting of baked goods; sweet bakery products, pre-made sweet bakery mixes for preparing sweet bakery products; pie fillings and other sweet fillings, gelatins and puddings; frozen desserts; yogurts; snack bars; bread products; pre-made bread mixes for preparing bread products; sauces, syrups and dressings; sweet spreads; confectionary products; and sweetened breakfast cereals.

18. The composition of claim 16, wherein the product for oral administration is a beverage product selected from the group consisting of carbonated beverages; non-carbonated beverages;
and beverage concentrates.

19. The composition of claim 2 or 3, wherein the truffle of family Terfeziaceae comprises a truffle of genus Terfezia, Tirmania, or Mattirolomyces.
